(19) United States
(12) Patent Application Publication
(10) Pub. No.: US 2017/0311591 A1
(43) Pub. Date: Nov. 2, 2017

(54) TOPICAL CONJUGATES AND COMPOSITIONS

(71) Applicant: NEO-INNOVA HEALTHCARE LIMITED, Hook, Hampshire (GB)

(72) Inventors: John Hywel DAVIES, Hook (GB); Christopher Adrian WILLIAMS, Reading (GB)

(21) Appl. No.: 15/524,813

(22) PCT Filed: Nov. 6, 2015
(86) PCT No.: PCT/EP2015/075991

§ 371 Date: May 5, 2017

(30) Foreign Application Priority Data
Nov. 6, 2014 (GB) 1419794.1

(51) Int. Cl. A01N 31/06; A61K 8/73; A61K 8/34; A61Q 17/02; A01N 25/18

(52) U.S. Cl. CPC A01N 31/06; A61Q 17/02; A01N 25/18; A61K 8/345; A61K 8/735

(57) ABSTRACT

The present invention relates to a conjugate suitable for topical application to the skin of a user, the conjugate comprising at least one volatile active coupled directly or indirectly to a carrier substance, wherein the carrier substance is substantially resistant to permeation through the skin of a user under ambient conditions, and wherein the coupling between the active and the carrier substance is such that said coupling is cleaved under said ambient conditions when said conjugate has been applied to the skin of a user, so as to release said volatile active from said carrier substance in a sustained release manner. The present invention also relates to a composition including such a conjugate and also processes of preparing such conjugates and compositions.

The present invention relates to topical conjugates and compositions based on at least one volatile active coupled directly or indirectly to a carrier substance, which carrier substance is substantially resistant to permeation through the skin of a user.

The present invention has particular utility in the area of insect repellents for use in repelling flies in particular of the family Culicidae or Ceratopogonidae, especially mosquitos and midges (hereafter collectively described as “biting flies”), whereby a selected volatile repellent is released from a conjugate or composition as applied to the skin of a user in a sustained release fashion and thus over a prolonged period of time. The present invention also has utility in the area of personal fragrances, whereby a selected volatile fragrance is similarly released from a conjugate or composition as applied to the skin of a user in a sustained release fashion and thus over a prolonged period of time.

According to the present invention, conjugates and compositions are provided that generate long acting and sustained release of volatiles that have been applied to the skin of a user. In particular for such conjugates and compositions based on insect repellents, the present invention can provide low cost biting fly repellents from natural, or synthetic products, or a mixture thereof. Such repellents are advantageous because they will lessen the number of bites received by a human, mammal, or other animal to which the repellent is applied and so increase the comfort of the human, mammal, or other animal to which the repellent is applied. In the case of biting flies that act as a vector for disease, for example mosquitos acting as a vector for disease, for example, malaria, dengue, West Nile virus, chikungunya, yellow fever, Japanese encephalitis, St Louis encephalitis, Western equine encephalitis, Venezuelan equine encephalitis, Eastern equine encephalitis and/or La Crosse virus, repelling said flies from biting humans, mammals, or other animals to which the repellent is applied will help reduce the incidence and spread of disease.

The insect repellent conjugates or compositions of the present invention provide personal protection for a human, mammal, or other animal to which the repellent is applied and may be tailored to repel biting flies such as, but not limited to, mosquitos in differing territories. This is because different species of biting flies are optimally repelled by different insect repellents.

According to a first embodiment of the present invention, there is provided a conjugate suitable for topical application to the skin of a user, the conjugate comprising at least one volatile active coupled directly or indirectly to a carrier substance, wherein the carrier substance is substantially resistant to permeation through the skin of a user under ambient conditions, and wherein the coupling between the active and the carrier substance is such that said coupling is cleaved under said ambient conditions when said conjugate has been applied to the skin of a user, so as to release said volatile active from said carrier substance in a sustained release manner.

According to a second embodiment of the present invention, there is provided a composition suitable for topical application to the skin of a user, the composition comprising a conjugate and at least one inert diluent or excipient therefor, said conjugate comprising at least one volatile active coupled directly or indirectly to a carrier substance, wherein the carrier substance is substantially resistant to permeation through the skin of a user under ambient conditions, and wherein the coupling between the active and the carrier substance is such that said coupling is cleaved under said ambient conditions when said conjugate has been applied to the skin of a user, so as to release said volatile active from said carrier substance in a sustained release manner.

According to a third embodiment of the present invention, there is provided a conjugate for releasing at least one volatile active in a sustained release manner when said conjugate has been applied to the skin of a user, the conjugate comprising the at least one volatile active coupled directly or indirectly to a carrier substance, wherein the carrier substance is substantially resistant to permeation through the skin of a user under ambient conditions, and wherein the coupling between the active and the carrier substance is such that said coupling is cleaved under said ambient conditions when said conjugate has been applied to the skin of a user. This third aspect of the present invention applies equally to a composition as described herein.

More specifically, according to the third embodiment of the present invention, there is provided a conjugate for repelling insects, wherein said conjugate releases at least one volatile insect repellent in a sustained release manner when said conjugate has been applied to the skin of a user, the conjugate comprising at least one volatile insect repellent coupled directly or indirectly to a carrier substance, wherein the carrier substance is substantially resistant to permeation through the skin of a user under ambient conditions, and wherein the coupling between the insect repellent and the carrier substance is such that said coupling is cleaved under said ambient conditions and when said conjugate has been applied to the skin of a user.

Also according to the third embodiment of the present invention, there is provided a conjugate for providing a desirable fragrance, wherein said conjugate releases at least one volatile fragrance in a sustained release manner when said conjugate has been applied to the skin of a user, the conjugate comprising at least one volatile fragrance coupled directly or indirectly to a carrier substance, wherein the carrier substance is substantially resistant to permeation through the skin of a user under ambient conditions, and wherein the coupling between the fragrance and the carrier substance is such that said coupling is cleaved under said ambient conditions and when said conjugate has been applied to the skin of a user.

According to a fourth embodiment of the present invention, there is provided a method of releasing at least one volatile active in a sustained release manner when said active has been applied to the skin of a user, wherein said active is present in a conjugate and said conjugate comprises the at least one volatile active coupled directly or indirectly to a carrier substance, wherein the carrier substance is substantially resistant to permeation through the skin of a user under ambient conditions, wherein said method comprises the steps of:
applying the conjugate to the skin of the user;
effecting cleavage of the coupling between the active and the carrier substance, so that said sustained release of said active occurs.

This fourth aspect of the present invention applies equally to a composition as described herein.

The term “volatile active” as referred to herein denotes a substance that when applied to the skin of a user, and the coupling between the active and its carrier substance is cleaved, then the active easily evaporates at ambient temperatures. The active can then provide an odorant vapour layer or vapour zone (for example, be this an odorant that provides insect repellence, or an odorant that is a desirable fragrance to a human user) adjacent the skin of the user.

The term “ambient conditions” as referred to herein denotes the immediate surroundings of a user of a conjugate or composition of the present invention, and in particular relates to the surrounding temperature. As will be appreciated the surrounding or ambient temperature will depend on the location of a user and if appropriate a conjugate or a composition according to the present invention can be tailored to be favourably adapted for a proposed location of use. For example, a conjugate or composition intended for use in a tropical environment, might require more volatile active therein so as to achieve the desired sustained release of the present invention over a prolonged period of time.

The terms “coupled” or “coupling” as used herein denote a direct or indirect attachment between the volatile active and the carrier substance. Thus the volatile active can be directly attached to the carrier substance based on a suitable bonding therebetween as hereinafter described in greater detail. Alternatively, the volatile active can be indirectly attached to the carrier substance by way of an appropriate linker. In either embodiment however, the coupling is such that this can be cleaved under ambient conditions when the conjugate or composition of the present invention has been applied to the skin of a user.

The term “sustained release” as used herein denotes a prolonged release period preferably of at least 6 hours, more preferably at least 8 hours, more preferably at least 9 hours, more preferably at least 10 hours, more preferably at least 11 hours and in some instances most preferably for at least 12 hours. It can be appreciated that in particular for the embodiments of the present invention wherein the volatile active comprises an insect repellent, then such a release profile can provide protection for a user against biting flies throughout a period of sleep without the need for reapplication, and in particular can provide protection for a user against biting flies throughout the night. Furthermore, such sustained release is clearly also advantageous in terms of providing prolonged daytime protection against biting flies. In particular for example, the Aedes aegypti mosquito is a day time biter, and a conjugate or composition according to the present invention can advantageously also provide prolonged protection against the same during the day as a result of the sustained release associated with such a conjugate or composition according to the present invention.

The term “carrier substance” as used herein denotes a substance which is substantially resistant to permeation through the skin of a user under ambient conditions. For example, if a carrier substance of sufficient molecular weight is selected, there will be little permeation through the skin of a user. Additionally or as an alternative proposal, the carrier substance can be selected so as to be sufficiently hydrophilic, which again should increase resistance to permeation through the skin by the carrier substance.

As is known in the art of topical administration, the skin protects against the influx of toxins and the efflux of water and is largely impermeable to the penetration of foreign molecules. Human skin consists of three main layers: the epidermis, dermis, and hypodermis. The epidermis, in particular the stratum corneum, acts as the major barrier to penetration. The stratum corneum is a highly lipophilic membrane; it is 10-20 pm thick depending on its state of hydration. The thickness of the epidermis varies from 0.06 mm on eyelids to 0.8 mm on the soles of the feet.

An applied agent that permeates the skin by way of transdermal administration must traverse these structural layers, encountering several lipophilic and hydrophilic domains on the way to the dermis where absorption into the systemic circulation occurs. Generally speaking, a high molecular weight and/or hydrophilic agent will exhibit low permeation through the skin and in the present case it is preferred that at least the molecular weight of the carrier substance is selected so as to provide the required resistance to skin permeation.

Such reduction in skin permeation can also be beneficial in achieving a corresponding reduction in systemic absorption. Carrier substances of sufficient molecular weight permeate very poorly through human skin as explained above and so will reside in or on the skin, thus similarly reducing systemic absorption of potentially harmful foreign substances, such as an insect repellent when this is selected as the volatile active to be used in the present invention. This advantage should be contrasted to established small molecule insect repellents, such as DEET, that can more readily permeate through skin and thus can enter the systemic circulation.

There are also variations between individuals in the rate at which agents can penetrate the skin due to factors such as thickness of the stratum corneum, skin hydration, underlying skin diseases or injuries, ethnic differences, and body temperature. Such differences should also be taken into account when predicting a sustained release delivery period as referred to hereinbefore.

In terms of the volatile active as described herein, it will be appreciated that one preferred such active comprises an insect repellent. It will also be appreciated that an alternative preferred such active comprises a fragrance. Such actives are however given by way of example only, and are not intended as limiting on the scope of the present invention. For example, any volatile active that can be cleaved from a carrier molecule after application to the skin of a user and is then capable of easily evaporating and proving a vapour layer or vapour zone adjacent the skin of a user can be used in a conjugate or composition of the present invention.

A volatile active that can be used in the present invention includes at least one group that is capable of forming a bond with at least one group as present in the carrier substance. Typically a volatile active to be used in the present invention includes at least one OH group in its structure, whereby the active is thus particularly suited to form an ester bond with one or more carboxylic acid groups as present in a carrier substance as used in the present invention. Alternatively, a volatile active to be used in the present invention includes at least one carboxylic acid group in its structure, whereby the active is thus particularly suited to form an ester bond with one or more OH groups as present in a carrier substance as used in the present invention. Such a resulting ester bond (regardless of where the carboxylic acid/OH groups originate from) can be advantageously cleaved by natural esterases present in the skin of a user, or associated with bacteria overlying the skin of a user, thus liberating the volatile active over extended periods of time. It will of course be appreciated that different groups and resulting bonds may be present in the coupling as present in a conjugate or composition of the present invention, the only limiting factor being that such coupling can be cleaved when the conjugate or composition has been applied to the skin of a user.

In the case where the volatile active comprises an insect repellent, this may be one or more natural insect repellents, one or more synthetic insect repellents, or a mixture of natural and synthetic insect repellents. As described above, most preferably, the or each insect repellent includes at least one OH group in its structure.

The carrier substance to which the volatile active is coupled may be a single substance, or a mixture of different substances, all of which may bond with the volatile active. A particularly preferred carrier substance comprises one or more polymers, all of which may bond with the volatile active. Such polymers are preferably selected so as to provide the desirable molecular weight so as to provide resistance to skin permeation as hereinbefore described. Alternatively, the hydrophilicity of the carrier substance, preferably a polymer, may be selected so as to provide the resistance to skin permeation.

Suitable molecular weights of polymers to be used in the present invention so as to provide the desired resistance to skin permeation are usually greater than 1000 daltons, as measured by size exclusion chromatography, also commonly known as gel permeation chromatography. Typically, however, the molecular weights of polymers to be used in the present invention are less than 1.8 million daltons again as measured by size exclusion chromatography. Preferred molecular weight ranges can be 20,000 to 50,000 daltons, or 100,000 to 300,000 daltons, for a polymer to be used in the present invention. These molecular weights are number average molar mass.

Even more preferably, the present invention thus provides conjugates and compositions wherein a volatile active as described herein is directly coupled to a polymer carrier substance by way of an ester linkage, which can, in use, be advantageously cleaved by esterases. Other coupling means are of course envisaged within the scope of the present invention and for example the volatile active may be joined to the polymer by an amide bond, or by an ether bond, or by a ketone bond, or by an aldehyde bond. Whichever specific coupling means is selected, it is further preferred that the polymer is so structured that it is adsorbed or retained in or on the outer layer of the skin (the epidermis).

An advantage of the present invention is that where the polymer is so structured that it is adsorbed or retained in or on the outer layer of the skin as described above, then the conjugates or compositions of the present invention will be resistant to removal from the skin as a result of rubbing and/or sweating. Such premature removal of insect repellents as a result of rubbing and/or sweating was a problem known to be associated with prior art insect repellents, especially in tropical climates where users would often sweat profusely resulting in loss of the repellent from the skin. The present invention however solves this problem as associated with the prior art and reduces the need for reapplication of the conjugates and compositions of the present invention. In this way, the present invention increases the period of time during which the conjugates and compositions exhibit their desirable and efficacious utilities, for example an increase in the period of time over which insects are repelled from a user of a conjugate or composition of the present invention.

In particular in a conjugate or composition according to the present invention for use in repelling biting flies, such a conjugate or composition preferably comprises a mixture of two or more different polymer-insect repellent conjugates. Such a mixture can be advantageous in optimizing the insect repellent properties of a conjugate or composition according to the present invention.

A polymer carrier substance to be used in the present invention includes at least one group that is capable of forming a bond with at least one group as present in the volatile active. A preferred polymer carrier substance to be used in the present invention is selected from the group consisting of hyaluronic acid, polycarboxylic acids, other suitable carboxylic acids, and mixtures thereof. Most preferably, a polymer carrier substance to be used in the present invention is selected from the group consisting of hyaluronic acid, polycarboxylic acid, and mixtures thereof. The term “polycarboxylic acid” as used herein denotes any organic compound having multiple carboxylic acid functional groups.

Hyaluronic acid and/or polycarboxylic acids have the additional benefit of being well-established moisturizers and so will aid skin hydration. This is particularly beneficial in arid conditions whereby such a moisturising effect may improve application compliance among human users of the conjugates and compositions of the present invention.

As can be appreciated from the above discussion, a polymer carrier substance as used in the present invention typically includes one or more carboxylic acid groups that can form an ester bond with at least one OH group typically present in a volatile active. Alternatively, a polymer carrier substance as used in the present invention can include one or more OH groups (such as a polyvinyl alcohol) that can form an ester bond with at least one carboxylic acid group typically present in the volatile active. Such a resulting ester bond (regardless of where the carboxylic acid/OH groups originate from) can be advantageously cleaved by natural esterases present in the skin of a user, or associated with bacteria overlying the skin of a user, thus liberating the volatile active over extended periods of time. It will of course be appreciated that different groups and resulting bonds may be present in the coupling as present in a conjugate or composition of the present invention, the only limiting factor being that such coupling can be cleaved when the conjugate or composition has been applied to the skin of a user. For example, either the volatile active or the carrier substance can include one or more of the following organic compounds, namely polyesters, polyalcohols, polyamides, polyketones or the like, that can form bonds with the other component of the conjugate or composition and which bonds are cleavable in use as herein described.

In the case wherein the volatile active comprises an insect repellent as described herein, the insect repellent can be any insect repellent that is suitable for coupling to a carrier material as described herein, whilst also being capable of cleavage therefrom so as to demonstrate a sustained release profile also as hereinbefore described. Such insect repellents can include, but are not limited to, p-menthane-3,8-diol (PMD), one or more of the known insect repellent terpenes, such as geraniol or nerolidol, or one or more the known synthetic insect repellents, such as picaridin.

Such insect repellents or indeed any other suitable volatile actives as described herein can be attached to the carrier substance, such as a polymer carrier substance, by well-known chemical reactions. For example, an acid catalyzed dehydration of polymer carboxylic acid groups bonding with the alcohol of the volatile active forming an ester bond. The polymer-volatile active conjugates are preferably blended to provide a suite of compositions or formulations with tailored activity against biting flies, in particular mosquito strains in different regions of the world.

In preferred embodiments of the present invention, the insect repellents are therefore selected for their repellent properties against the key mosquito vectors in the transmission of malaria.

In other preferred embodiments of the present invention, the insect repellents are selected for their repellent properties against the key mosquito vectors in the transmission of dengue, West

Nile virus, chikungunya, yellow fever, Japanese encephalitis, St Louis encephalitis, Western equine encephalitis, Venezuelan equine encephalitis, Eastern equine encephalitis and/or La Crosse virus. In this way, the above described suite of compositions can be provided with tailored activity against biting flies in different regions of the world.

It will be appreciated that other chemical reactions can be used to bond the volatile active to the carrier substance, such as the polymer carrier substance, and from which the volatile active may be released over prolonged times. For example, the volatile active may be joined to the polymer by an amide bond, or by an ether bond, or by a ketone bond, or by an aldehyde bond as hereinbefore described. The reactions to form these bonds can progress by intermediate steps, for example by forming an acid chloride from the carboxylic acid to react with the volatile active to form an amide, or preparing organohalides to react with the alcohol functional groups in the active or carrier substance to produce ether bonds between the active or carrier substance.

The present invention also thus provides a process of providing a conjugate as described herein, which process comprises coupling at least one volatile active with a carrier substance, under conditions so as to provide a conjugate wherein the coupling between the at least one volatile active and carrier substance is such that it can be cleaved under ambient conditions and when said conjugate has been applied to the skin of a user. For example, in the case wherein the coupling is an ester linkage, then the conditions should be such as to facilitate the formation of such an ester linkage.

Coupling the volatile active and carrier substance can be performed by many different reactions that are well known.

One approach is to convert the carboxylic acid, commonly of the polymer carrier (for example hyaluronic acid), into a more reactive acid chloride by reacting the carboxylic acid with phosphorous trichloride, thionyl chloride or other suitable chlorinating reagent. The acid chloride species formed can then be used to react with the alcohol-group containing mosquito repellent or fragrance material to form an ester linkage.

Alternatively, the polymeric carrier can be directly coupled to the volatile active by reactions that are well known. For example, PMD or geraniol can be coupled to hyaluronic acid as follows, with reference to the structures thereof.

Para-menthane-3,8-diol (PMD):

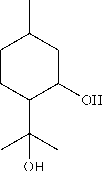

Geraniol:

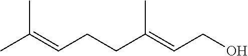

Hyaluronic acid:

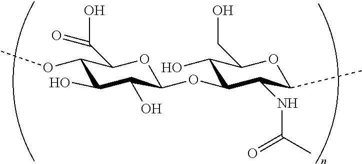

Based on the above structures, the following coupling can be carried out:

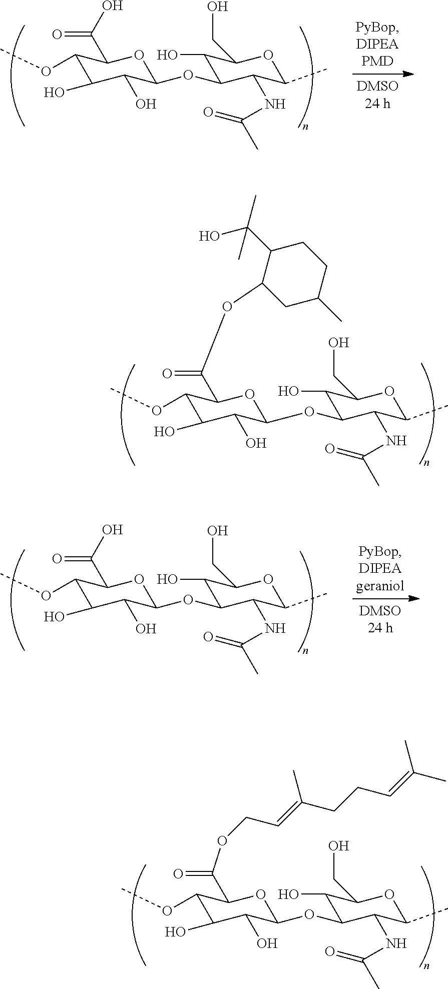

The above reaction steps are preferably carried out in the presence of a coupling agent. The reaction can preferably use PyBop as a coupling agent (PyBop is the common abbreviation for benzotriazol-1-yl-oxytripyrrolidinophosphonium hexafluorophosphate). Other suitable coupling agents that could be used in these reactions include benzotriazol-1-yloxy)tris(dimethylamino)phosphonium hexafluorophosphate which is commonly known as BOP or 3-(diethoxyphosphoryloxy)-1,2,3-benzotriazin-4(3H)-one which is commonly known as DEPBT or 1-[bis(dimethylamino)methylene]-1H-1,2,3-triazolo[4,5-b]pyridinium 3-oxid hexafluorophosphate which is commonly known as HATU or 2-(1H-benzotriazol-1-yl)-1,1,3,3-tetramethyluronium hexafluorophosphate which is commonly known as HBTU.

The above reaction steps are also preferably carried out in the presence of known esterification reagents. For example, DIPEA (N,N-diisopropylethylamine, also commonly known as Hünig's base) can preferably be used. Other well known bases can also be used in these reactions.

The above reaction steps are also preferably carried out in the presence of a polar aprotic solvent. DMSO (dimethyl sulphoxide) is a preferred such solvent, but other suitable solvents can also be used and could include, for example, dimethylformamide, dimethylacetamide or N-methyl-2-pyrrolidone.

The reaction can typically be performed at room temperature, or at slightly elevated temperatures, such as 35° C. The temperature of the reaction, as well as the precise coupling agent, solvent and base chosen will affect the time needed to allow the reagents to react, but the reaction will typically be completed within 24 hours.

Other reactions can be used to couple the volatile active to a carrier substance. A suitable alternative reaction includes the Pinner reaction, which is an organic reaction of a nitrile with an alcohol using an acid catalyst, for example, hydrochloric acid. The product formed is the hydrochloric acid salt of an imino ester or an alkyl imidate, which sometimes is called a Pinner salt. These salts can react with an excess of alcohol to form the orthoester or with water to form an ester.

According to the present invention therefore, using low cost materials and a simple “one-pot” synthetic methodology, an affordable conjugate, such as a polymer - insect repellent conjugate, can be produced. The resulting conjugate can exhibit prolonged activity of at least 6 hours, at least 8 hours, at least 9 hours, at least 10 hours, at least 11 hours or at least 12 hours as already hereinbefore described that can be particularly beneficial in terms of achieving prolonged insect repellence such as during the night. The present invention thus provides conjugates and compositions exhibiting improved insect repellent activity.

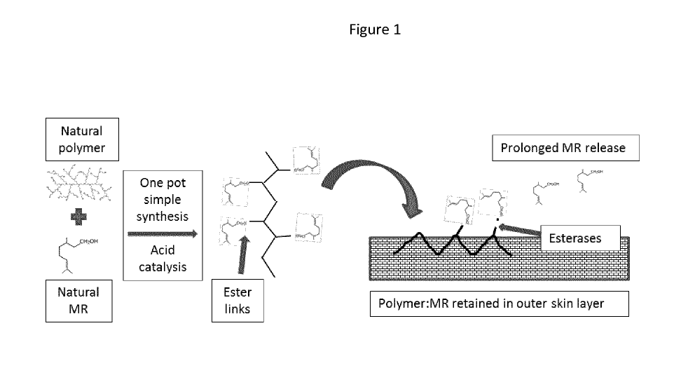

FIG. 1 illustrates a process of manufacturing a conjugate of the present invention and subsequent cleavage thereof in use.

A conjugate for use in repelling biting flies according to the present invention most preferably comprises one or more of:
a polymer-insect repellent conjugate where the polymer is chemically bonded to the insect repellent,
a polymer-insect repellent conjugate where the polymer is chemically bonded to the insect repellent, which is either a natural or a synthetic product, or a mixture thereof,
a polymer-insect repellent conjugate where the polymer is hyaluronic acid and the insect repellent is PMD,
a polymer-insect repellent conjugate where the polymer is hyaluronic acid and the insect repellent is a terpene,
a polymer-insect repellent conjugate where the polymer is hyaluronic acid and the insect repellent is geraniol,
a polymer-insect repellent conjugate where the polymer is hyaluronic acid and the insect repellent is nerolidol,
a polymer-insect repellent conjugate where the polymer is hyaluronic acid and the insect repellent is a synthetic repellent, such as picaridin,
a polymer-insect repellent conjugate where the polymer is a polycarboxylic acid and the insect repellent is PMD,
a polymer-insect repellent conjugate where the polymer is a polycarboxylic acid and the insect repellent is a terpene,
a polymer-insect repellent conjugate where the polymer is a polycarboxylic acid and the insect repellent is geraniol,
a polymer-insect repellent conjugate where the polymer is a polycarboxylic acid and the insect repellent is nerolidol,
a polymer-insect repellent conjugate where the polymer is a polycarboxylic acid and the insect repellent is a synthetic repellent, such as picaridin.

As hereinbefore described, a composition according to the present invention comprises a conjugate (also an embodiment of the present invention as hereinbefore described) and at least one inert diluent or excipient therefor. Such a composition according to the present invention can be in the form of creams, ointments, gels, solutions or suspensions, etc., comprising a conjugate as described herein. Such compositions generally further comprise at least an inert diluent or excipient, and possibly also one or more of a cosolvent, emulsifier, preservative system, and/or emollient. In some embodiments, the compositions may be sprayed on to the skin of a user. In other embodiments, and for more viscous compositions, such compositions may be rubbed onto the skin of a user, thereby providing a coating thereon. Particularly preferred compositions according to the present invention can be in the form of a gel, or a spray, for application to the skin of a user.

For example, suitable inert diluents or excipients can include water and/or one or more alcohols (such as ethanol) and/or one or more esters (such as isopropyl myristate).

For example, suitable preservatives can include polyquatemium-1, chloride, thimerosal, chlorobutanol, methyl paraben, propyl phenylethyl alcohol, edetate disodium, sorbic acid, or other agents known to those skilled in the art.

For example, suitable co-solvents can include polysorbate 20, 60, and 80, polyoxyethylene/polyoxypropylene surfactants (such as glycols or block copolymers), or other agents known to those skilled in the art.

In a particularly preferred embodiment, a composition according to the present invention can further comprise at least one further volatile active in addition to the at least one volatile active coupled directly or indirectly to a carrier substance as described herein. This coupled volatile active is now for this particular embodiment of the present invention and for ease of reference denoted as the “at least first volatile active”. The further volatile active may be the same or different to the first volatile active, but is preferable present in substantially non-bound form. In other words, the further volatile active is not part of a conjugate as present in a composition according to the present invention. In this way, the further volatile active can exhibit an immediate release profile thus providing an immediate effect for example either as an insect repellent or a desirable fragrance, whereas the at least first volatile active as present in the conjugate can exhibit a sustained release profile thus providing a prolonged effect for example either as an insect repellent or a desirable fragrance. This dual release profile can be particularly advantageous when the first and further volatile actives are insect repellents, and thus a composition according to the present invention can provide immediate and sustained protection against biting flies.

The present invention further comprises a process of preparing a composition according to the present invention, which process comprises admixing a conjugate as hereinbefore described with at least a suitable diluent or excipient so as to provide a composition according to the present invention. As hereinbefore described, wherein the volatile active comprises an insect repellent, the insect repellent can be selected according to territory and as such a most efficacious repellent for biting flies in the territory.

The present invention will now be further illustrated by the following Examples, which are not intended to limit the scope of the invention in any way.

### EXAMPLE 1

DOWEX ion exchange resin (12.5 g) was washed three times with 250 mL aliquots of distilled water and then treated with 25 mL of tetra-n-butyl ammonium hydroxide (TBA) and left to react at room temperature for 1 hour. The resin (Dowex-TBA) was then filtered and the resin collected. 1 g of hyaluronic acid (HA) was dissolved in 100 mL of distilled water to which 10 g of the DOWEX-TBA resin was added and the reaction was allowed to proceed at room temperature for 18 hours. The reaction mixture was then filtered through a 0.45 μm filter and the supernatant was collected. The supernatant was freeze dried for three days to yield HA-TBA. 500 mg of HA-TBA was dissolved in 60 mL dimethyl sulphoxide (DMSO). 0.5 mL of N,N-diisopropylethylamine (DIPEA) and 0.9 g of benzotriazol-1-yl-oxytripyrrolidinophosphonium hexafluorophosphate (PyBop) were added and left stirring at room temperature for 1 hour. 1 g of para-menthane-3,8-diol (PMD) was then added and the mixture left stirring for a further 24 hours at room temperature. The resultant HA-PMD conjugate was then recovered from the reaction mixture by dialysis against an excess amount of 100 mM sodium chloride aqueous solution containing 25% v/v ethanol and distilled water (5 changes over 48 hours) before freeze drying for 3 days to provide the solid HA-PMD conjugate.

### EXAMPLE 2

The HA-PMD conjugate was formulated into a hydroalcoholic gel. Deionised water (47 weight %), denatured ethanol (31.75 weight %), polyethylene glycol with an average molecular weight of 400 Da (10 weight %), 0.5M aqueous sodium hydroxide (0.25 weight %) were mixed until uniform at room temperature. To this mixture was added the HA-PMD conjugate (10 weight %) prepared according to Example 1 and Carbopol Ultrez 10 polymer (1 weight %). The mixture was stirred at 1200 rpm until a clear gel is obtained.

### EXAMPLE 3

The HA-PMD conjugate was formulated into a topical spray. Isopropyl myristate (30 weight %), propylene glycol (15 weight %), distilled water (10 weight %) and denatured ethanol (35 weight %) were mixed until uniform at room temperature. To this mixture was added the HA-PMD conjugate (10 weight %) prepared according to Example 1 and the mixture was stirred until uniform. The resultant formulation was packaged into a spray delivery system.

1. A conjugate suitable for topical application to the skin of a user, the conjugate comprising at least one volatile active coupled directly or indirectly to a carrier substance, wherein the carrier substance is substantially resistant to permeation through the skin of a user under ambient conditions, and wherein the coupling between the active and the carrier substance is such that said coupling is cleaved under said ambient conditions when said conjugate has been applied to the skin of a user, so as to release said volatile active from said carrier substance in a sustained release manner.

2. A composition suitable for topical application to the skin of a user, the composition comprising a conjugate and at least one inert diluent or excipient therefor, said conjugate comprising at least one volatile active coupled directly or indirectly to a carrier substance, wherein the carrier substance is substantially resistant to permeation through the skin of a user under ambient conditions, and wherein the coupling between the active and the carrier substance is such that said coupling is cleaved under said ambient conditions when said composition has been applied to the skin of a user, so as to release said volatile active from said carrier substance in a sustained release manner.

3. A conjugate for releasing at least one volatile active in a sustained release manner when said conjugate has been applied to the skin of a user, the conjugate comprising the at least one volatile active coupled directly or indirectly to a carrier substance, wherein the carrier substance is substantially resistant to permeation through the skin of a user under ambient conditions, and wherein the coupling between the active and the carrier substance is such that said coupling is cleaved under said ambient conditions when said conjugate has been applied to the skin of a user.

4. A composition for releasing at least one volatile active in a sustained release manner when said composition has been applied to the skin of a user, the composition comprising a conjugate and at least one inert diluent or excipient therefor, said conjugate comprising the at least one volatile active coupled directly or indirectly to a carrier substance, wherein the carrier substance is substantially resistant to permeation through the skin of a user under ambient conditions, and wherein the coupling between the active and the carrier substance is such that said coupling is cleaved under said ambient conditions when said composition has been applied to the skin of a user.

5. A conjugate for repelling insects, wherein said conjugate releases at least one volatile insect repellent in a sustained release manner when said conjugate has been applied to the skin of a user, the conjugate comprising the at least one volatile insect repellent coupled directly or indirectly to a carrier substance, wherein the carrier substance is substantially resistant to permeation through the skin of a user under ambient conditions, and wherein the coupling between the insect repellent and the carrier substance is such that said coupling is cleaved under said ambient conditions when said conjugate has been applied to the skin of a user.

6. A composition for repelling insects, wherein the composition comprises a conjugate and at least one inert diluent or excipient therefor, said conjugate comprising at least one volatile insect repellent coupled directly or indirectly to a carrier substance, wherein the carrier substance is substantially resistant to permeation through the skin of a user under ambient conditions, and wherein the coupling between the insect repellent and the carrier substance is such that said coupling is cleaved under said ambient conditions when said composition has been applied to the skin of a user, so as to release said volatile insect repellent from said carrier substance in a sustained release manner.

7. A conjugate for providing a desirable fragrance, wherein said conjugate releases at least one volatile fragrance in a sustained release manner when said conjugate has been applied to the skin of a user, the conjugate comprising at least one volatile fragrance coupled directly or indirectly to a carrier substance, wherein the carrier substance is substantially resistant to permeation through the skin of a user under ambient conditions, and wherein the coupling between the fragrance and the carrier substance is such that said coupling is cleaved under said ambient conditions when said conjugate has been applied to the skin of a user.

8. A composition for providing a desirable fragrance, wherein the composition comprises a conjugate and at least one inert diluent or excipient therefor, said conjugate comprising at least one volatile fragrance coupled directly or indirectly to a carrier substance, wherein the carrier substance is substantially resistant to permeation through the skin of a user under ambient conditions, and wherein the coupling between the fragrance and the carrier substance is such that said coupling is cleaved under said ambient conditions when said composition has been applied to the skin of a user, so as to release said volatile fragrance from said carrier substance in a sustained release manner.

9. A method of releasing at least one volatile active in a sustained release manner when said active has been applied to the skin of a user, wherein said active is present in a conjugate and said conjugate comprises the at least one volatile active coupled directly or indirectly to a carrier substance, wherein the carrier substance is substantially resistant to permeation through the skin of a user under ambient conditions, wherein said method comprises the steps of:
applying the conjugate to the skin of the user;
effecting cleavage of the coupling between the active and the carrier substance, so that said sustained release of said active occurs.

10. A method according to claim 9, wherein said conjugate is present in a composition that further comprises at least one inert diluent or excipient for said conjugate.

11. A conjugate according to claim 1, a composition according to claim 2, a method according to claim 9 or 10, wherein said volatile active comprises an insect repellent.

12. A conjugate according to claim 1, a composition according to claim 2, a method according to claim 9 or 10, wherein said volatile active comprises a desirable fragrance.

13. A conjugate according to any of claims 1, 3, 5, 7; a composition according to any of claims 2, 4, 6, 8; a method according to claim 9 or 10; wherein said sustained release is a release period of at least 6 hours, more preferably at least 8 hours, more preferably at least 9 hours, more preferably at least 10 hours, more preferably at least 11 hours and in some instances most preferably for at least 12 hours.

14. A conjugate, composition or method according to claim 13, wherein the release profile provides protection for a user against biting flies throughout a period of sleep without the need for reapplication.

15. A conjugate, composition or method according to claim 14, wherein the release profile provides protection for a user against biting flies throughout the night.

16. A conjugate, composition or method according to preceding claims, wherein the volatile active, or insect repellent, or fragrance, includes at least one OH group in its structure.

17. A conjugate, composition or method according to claim 16, wherein the volatile active, or insect repellent, or fragrance, forms an ester bond with one or more carboxylic acid groups as present in the carrier substance.

18. A conjugate, composition or method according to preceding claims, wherein the carrier substance comprises one or more polymers.

19. A conjugate, composition or method according to claim 18, wherein the polymer is selected from the group consisting of hyaluronic acid, polycarboxylic acids, other suitable carboxylic acids, and mixtures thereof.

20. A conjugate, composition or method according to claim 19, wherein the polymer is selected from the group consisting of hyaluronic acid, polycarboxylic acid, and mixtures thereof.

21. A conjugate, composition or method according to preceding claims, wherein the volatile active or volatile insect repellent is selected from the group consisting of p-menthane-3,8-diol, geraniol, nerolidol and picararidin.

22. A composition according to preceding claims, which further comprise at least one further volatile active in addition to the at least one volatile active coupled directly or indirectly to the carrier substance.

23. A composition according to claim 22, wherein the further volatile active is the same as the at least one volatile active coupled directly or indirectly to the carrier substance.

24. A composition according to claim 22, wherein the further volatile active is different to the at least one volatile active coupled directly or indirectly to the carrier substance.

25. A composition according to claims 22 to 24, wherein said at least one further volatile active is present in substantially non-bound form.

26. A composition according to claim 25, wherein said at least one further volatile active exhibits an immediate release profile, whereas the at least one volatile active coupled directly or indirectly to the carrier substance exhibits a sustained release profile.

27. A composition according to any of claims 22 to 26, wherein said at least one further volatile active, and said at least one volatile active coupled directly or indirectly to the carrier substance, are both insect repellents.

28. A composition according to claim 27, wherein said at least one volatile active coupled directly or indirectly to the carrier substance is selected from the group consisting of p-menthane-3,8-diol, geraniol, nerolidol and picararidin

29. A process of providing a conjugate according to claim 1, which process comprises coupling at least one volatile active with a carrier substance, under conditions so as to provide a conjugate wherein the coupling between the at least one volatile active and carrier substance is such that it can be cleaved under ambient conditions and when said conjugate has been applied to the skin of a user.

30. A process according to claim 29, wherein the reaction is carried out in the presence of a coupling agent.

31. A process according to claim 30, wherein the coupling agent comprises PyBop.

32. A process according to any of claims 29 to 31, wherein the reaction is carried out in the presence of an esterification reagent.

33. A process according to claim 32, wherein the esterification reagent comprises DIPEA.

34. A process according to any of claims 29 to 33, which is carried out in the presence of a polar aprotic solvent.

35. A process according to claim 34, wherein the solvent comprises DMSO.

36. A process of preparing a composition according to claim 2, which process comprises admixing a conjugate according to claim 1 with at least a suitable diluent or excipient so as to provide a composition according to claim 2.
